(19) United States
(12) Patent Application Publication
(10) Pub. No.: US 2019/0371477 A1
(43) Pub. Date: Dec. 5, 2019

(54) SYSTEMS FOR MAKING PERSONALIZED MEDICINE

(71) Applicant: Vitae Industries, Inc., Providence, RI (US)

(72) Inventors: Daniel DeCiccio, Providence, RI (US); Jeanine Sinanan-Singh, Providence, RI (US)

(21) Appl. No.: 16/439,077

(22) Filed: Jun. 12, 2019

Related U.S. Application Data
(63) Continuation of application No. 15/162,168, filed on May 23, 2016.
(60) Provisional application No. 62/173,469, filed on Jun. 10, 2015.
(60) Provisional application No. 62/165,944, filed on May 23, 2015.

(51) Int. Cl. G16H 70/60; G16H 50/20

(52) U.S. Cl. CPC G16H 70/60; G16H 50/20

(57) ABSTRACT

A method for providing personalized medicine comprises the steps of: aggregating information regarding at least one medical topic of interest, the information relating to at least one of: the standard of care; best practices; proper dosage; and proper procedures; extracting and filtering to partition the information regarding the at least one medical topic of interest; and delivering a subset of the information filtered to a healthcare professional based upon the extracting and filtering. The information may include medical studies and medical literature. The method can instruct a physician on developing a treatment plan for a patient based on the subset.

### CROSS-REFERENCE TO RELATED APPLICATION

This application claims priority to U.S. Provisional Patent Application No. 62/173,469, filed Jun. 10, 2015, and U.S. Provisional Patent Application No. 62/165,944, filed May 23, 2015, which are incorporated herein by reference.

### FIELD

The subject disclosure relates to fabricating medicine and, more particularly, to systems and methods for fabricating medicine and/or supplements that are customized based on personal information.

### BACKGROUND

The supplement (or “nutraceutical”) market, while continuing to grow 5-7% annually, is still largely a commodity market, containing many products with little-to-no differentiation, added value for the customer, or customization. Consequently, competition is largely based on price for standard doses that customers often do not understand.

For example, although Internet searches for vitamin D are up 250% based on Google search indices, indicating growing interest and awareness, only 29% of existing vitamin users say they take vitamin D. Over 70% of Americans are vitamin D deficient. The vast majority of people who are vitamin D deficient have a detrimental unmet need. According to the Harvard School of Public Health, vitamin D deficiency has been linked to higher rates of cancer, cardiovascular disease and several other chronic and infectious diseases. Although it is a widespread problem, Americans only have access to a few standard doses that may, or may not, contain what they need.

Dietary supplements and medications are one size fits all, but individual health requirements are unique. Both over dosing and under dosing can cause harm, including side effects and decreased efficacy. For example, too much vitamin D can cause calcification of the organs. Getting just the right amount of vitamin D can be extremely difficult using existing technology and requires frequent blood tests and doctor visits.

Turning to other types of medication regimes, adherence to the regime is required for successful treatment outcomes. Pediatric adherence to prescribed medication is particularly difficult at only about 58% in one study. A sizeable proportion of children aged 4 to 12 are simply unable to swallow pills, even after behavioral training and rewards. Taste is the most commonly reported obstacle, affecting up to 35% of prescribed oral formulations and accounting for a 65% reduction in compliance. Low medication compliance endangers children's health and increases the cost of their hospital care.

The wide range of doses required for children based on age, weight and other factors further complicate pediatric drugs. As a result, liquid formulations are the most common alternate dosage form offered by both manufacturers and compounding pharmacists. However, both organoleptic and physical properties of custom liquid formulations still prevent high rates of compliance. Even worse, errors in preparation can result in wrong drug doses and even no drug dose. Imprecise measuring devices used by care givers, such as dinner teaspoons, also reduce accuracy. Some 15% or more of pediatric out-patients may receive incorrect doses. For example, prednisolone is widely prescribed yet largely rejected by children due to taste.

There is a large market for palatable custom drug formulations for children. Patients under 18 years old account for 11.5% of compounding pharmacy revenue—over $900 million—among the nearly 8000 compounding pharmacy establishments in the United States. Manual extemporaneous preparation of gummy dosage forms currently takes at least one hour of hands-on labor. Hence, although usage of such methods may improve compliance, adoption has been slow due to the inefficiency.

### SUMMARY

There is a pressing need to offer improved, traceable, and verifiable palatable formulations for patients and, in particular, pediatric patients.

The subject technology relates to methods and systems for personalized medicine which provides convenient, personalized value for each patient or individual. One benefit is the simplification of health decisions. The subject technology also provides economical custom dosing. The personalized supplement or medicine creates significant value in the undifferentiated vitamin supplement or compounding pharmacy businesses. In one instance, vitamin D or iron is the particular supplement.

The system and methods provide a convenient, effective way for an individual or patient to get just the right amount of a vitamin or medication. In particular, the subject technology avoids problems associated with improper doses and removes uncertainty by offering a personalized dietary supplement to help each individual achieve his or her optimal health. The personalized supplement provides individually tailored doses to keep the individual healthy. In one example, the method and system determines an individual's ideal dose of vitamin D for each month and provides the individual a monthly set of convenient, personalized daily pills to remedy deficiencies. In another embodiment, the subject technology provides a complete multivitamin supplement and/or medications.

In one embodiment, the method and system includes the steps of gathering information and providing that information to medical professionals to improve the quality of healthcare provided to patients or individuals. This method gathers medical information and data on a specific topic, or on various topics, and shares information with at least one medical professional. The method first aggregates medical information and data, such as medical studies, information from medical journals, information from medical websites, and other medical literature, and combines/augments the gathered data with raw, transformed and other scientific data. The information relates to at least one topic of interest, such as a standard of care; best medical practices; proper dosage of a pharmaceutical drug, vitamin, or other medication; and proper medical procedures. The method extracts the latest information regarding the topic of interest, or the topics of interest. The method then delivers the latest information to the healthcare professional requesting the information on the topic of interest. The method can also deliver this latest information to a database or a memory component of a computer or another electronic media storage device.

In another embodiment, a method and system includes a method for calculating and dispensing the appropriate amount of a pharmaceutical drug or vitamin or medication to a patient based on a patient health profile. This method prompts a patient or individual for health information and other personal information, and then calculates the appropriate amount of the pharmaceutical drug, vitamin, or other medication to dispense to the patient. The method prompts a patient to input health data related to the patient on a digital platform. The method then collects the health data of the patient and creates a health profile for that patient. Then, the method calculates the proper dose of a medical product for that patient based on the health profile of the patient, and based on at least one property of the medical product. The medical product can be, for example, a pharmaceutical drug, a vitamin, a medication, or another product. The method then communicates the proper dose to a dispenser, such as a 3D printer. The dispenser provides the proper dose of the medical product to the patient. Preferably, the method is executed on specialized hardware that includes easy to use buttons and interfaces that execute the process. For example, a series of buttons may be provided so that a first button starts the process and additional buttons perform each step of the process.

In another embodiment, the latest medical information and data are aggregated, and a health profile for a patient is created based on information gathered from the patient. The method calculates the appropriate amount of pharmaceutical drug, vitamin, or medication to dispense based on the medical information and data aggregated and based on the health profile of the patient. In this embodiment, the personalized dosage algorithm for calculating a dose can be updated, in some cases automatically, in response to the latest information collected by the method of the first embodiment. The method may also instruct a physician or other medical professional on developing a treatment plan for the patient.

The subject disclosure may also be embodied in a digital platform, which diagnoses and customizes vitamin D or other doses for both professionals and patients. The resulting treatment can include a variety of essential vitamins and minerals as well. The subject technology includes professional options for doctors and health care professionals, and provides similar app-based diagnostic tools for various prescription drugs where correct dosing is essential to efficacy such as antibiotics for acne or stimulants for attention deficit hyperactivity disorder (ADHD). A modular personalized production platform, such as with the envisioned specialized hardware including a 3D printer, offers doctors and pharmacies the ability to provide direct custom medication and supplement dosing to their patients. Other vitamins and supplements include, but are not limited to Vitamin K, Iron and Vitamin A as well as prescription medications and the like.

Accordingly, among the objects of the instant disclosure are: providing a method and/or system for improving the quality of care provided by medical professionals to patients; providing a method and/or system for aggregating the latest medical information and data regarding a topic of interest, and delivering that information to a doctor; providing a method and/or system for calculating the proper dose of a medical product for a patient, and dispensing the proper dose of the medical product to the patient; providing a method and/or system for aggregating the latest medical information regarding a topic of interest, acquiring information regarding a patient profile, using the latest medical information, data and patient profile to calculate the proper dose of a medical product for the patient, and dispensing the proper dose of the product to the patient; and providing a method and/or system for educating medical professionals regarding the standard of care, best practices, proper dosages, and proper medical procedures.

The subject technology addresses the critical unmet need for flexible doses and palatable formulations for children particularly well. By using a 3D printing process, pediatric drugs can be prepared quickly as easily chewable and palatable familiar forms such as “gummy bears.” The 3D printing of pediatric medications can accommodate the variety of doses and dosage forms required to effectively treat children, especially given the comparatively smaller pediatric market. While custom compounded solid oral dosage forms may be available, these forms are labor-intensive and expensive to make. Thus, compounded solid oral drugs are uncommon and largely focused on capsules. Further, the use of 3D printing in the pharmacy can provide efficiency and automation of manufacturing at a small scale required for individualizing solid oral medications. Still further, improving the ease of access to custom dose would facilitate large-scale studies to evaluate custom doses, and would support true dose titration for a patient. For children, it is particularly useful for the dose to be a familiar and palatable texture and size. Custom flavoring can further increase adherence and improve medical outcomes. The pediatric formulations can also be adjusted to reduce sugar content, which is an important concern especially for chronic dosing.

In another embodiment, generic synthetic glucocorticoid is formulated in a gummy dosage. Generic synthetic glucocorticoid is used to treat a variety of allergic disorders, skin conditions, ulcerative colitis, arthritis, lupus, psoriasis, and asthma. Up to 80% of pediatric patients reject the taste of prednisolone as soluble tablets or oral suspensions. Prednisolone usually requires large doses (as much as 80 mg/kg/day) which creates a large volume or pill burden that further decreases compliance. The palatable, flexibly dosed prednisolone gummy bear resolve these problems and positively impact pediatric adherence.

Preferably, the system including the 3D printer produces a batch of gummies in less than 15 minutes in an automated process. Custom flavoring provides economic benefits to the pharmacy through increased customer satisfaction, likelihood of recommendation, and/or an associated upcharge. One survey found that 42% of customers were more likely to switch to a pharmacy that offered custom flavors for their child's prescriptions and nearly 90% were willing to pay a small fee (up to $5) to customize taste.

One embodiment is a method for providing personalized medicine including the steps of: aggregating information regarding at least one medical topic of interest, the information relating to at least one of: the standard of care; best practices; proper dosage; and proper procedures; extracting and filtering to partition the information regarding the at least one medical topic of interest; and delivering a subset of the information filtered to a healthcare professional based upon the extracting and filtering. The information may include medical studies and medical literature. The method can instruct a physician on developing a treatment plan for a patient based on the subset.

Another method also provides personalized dosage of medicine by prompting an individual to input health data, collecting the health data related to the respective individual to create a health profile for the respective individual, calculating a proper dose of a medical product for the respective individual based on the health profile of the respective individual and based on at least one property of the medical product, communicating the proper dose to a dispenser, and instructing the dispenser to provide the dose to the respective individual. Preferably, the dispenser includes a 3D printer. The method may also aggregate information regarding at least one medical topic of interest, the information relating to at least one of: the standard of care; best practices; proper dosage; and proper procedures, and extract and filter the information by date regarding the topic of interest, wherein the step of calculating the proper dose of the medical product relies on a subset of the information filtered by date to modify coefficients used in calculating the proper dose.

Still another method provides personalized dosage of a product by collecting data related to an individual to create a profile for the respective individual, calculating a proper dose of a product for the respective individual based on the profile of the respective individual and based on at least one property of the product, and communicating the proper dose to a dispenser. The method may fit the profile to a mathematical model based on clinical studies. The method may also gather relevant data from a personal fitness device, analyzing the relevant data to determine an adjustment, and recalculating the proper dose based upon the adjustment. The relevant data is selected from geographic data, a dosimeter reading, and/or a pedometer reading. The method can also adjust at least one of a size and a shape based upon the proper dose.

It should be appreciated that the subject technology can be implemented and utilized in numerous ways, including without limitation as a process, an apparatus, a system, a device, a method for applications now known and later developed or a computer readable medium. It is also envisioned that particular hardware suited to provide the modules, as would be appreciated herein, can be provided to implement the subject technology. These and other unique features of the systems and methods disclosed herein will become more readily apparent from review of the subject disclosure. Other objects, features and advantages of the disclosure shall become apparent as the description thereof proceeds when considered in connection with the accompanying illustrative drawings.

### DESCRIPTION OF THE DRAWINGS

So that those having ordinary skill in the art to which the disclosed technology appertains will more readily understand how to make and use the same, reference may be had to the following drawings.

FIG. 1 is a block diagram of an environment with a delivery system embodying and implementing the methodology of the subject disclosure.

FIG. 2 is a schematic representation of the 3D printer in accordance with the subject technology.

FIG. 3 is a flowchart depicting a research process for finding the most pertinent information for health care providers and patients in accordance with the present technology.

FIG. 4 is a flowchart depicting a questionnaire process for determining a recipe for a medication in accordance with the present technology.

FIG. 5 is an exemplary results page for a process of the flowchart of FIG. 4.

FIG. 6 is a flowchart depicting a fabrication process for a customized medication in accordance with the present technology.

### DESCRIPTION OF THE EMBODIMENTS

The subject technology overcomes many of the prior art problems associated with providing medicine and/or supplements to individuals by customizing the deliverable based upon individual information in a health profile. The advantages, and other features of the systems and methods disclosed herein, will become more readily apparent to those having ordinary skill in the art from the following detailed description of certain preferred embodiments taken in conjunction with the drawings which set forth representative embodiments of the present invention and wherein like reference numerals identify similar structural elements.

In brief overview, personalized medicine in accordance with the subject technology improves the quality of medical care by providing updated, relevant information to physicians or other medical professionals, and by dispensing or enabling the dispensing of the proper dose of a medical product (such as a pharmaceutical drug, vitamin, etc.) tailored to a patient. For children, the subject technology provides an enhanced method for fabricating doses and an enhanced product so that compliance is greatly improved among other benefits.

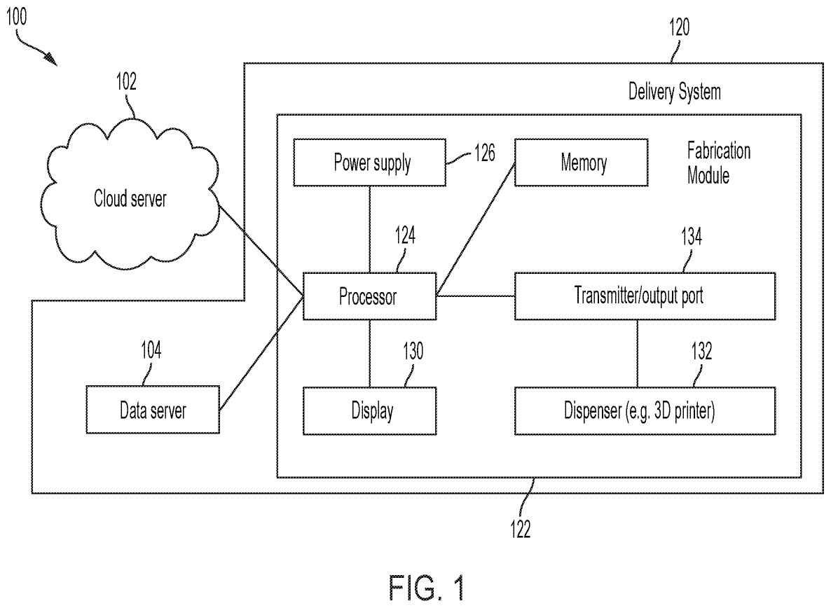

Referring now to the FIG. 1, there is shown a block diagram of an environment 100 with a delivery system 120 embodying and implementing the methodology of the present disclosure. The environment 100 connects users (e.g., healthcare professionals, pharmacists, and patients) with a cloud server 102 via the delivery system 120 for fabrication of customized medicine and supplements. The delivery system 120 is user-interactive and may be self-contained so that users need not leave or venture to another physical location or virtual address within a distributed computing network to access various information and produce various items. The following discussion describes the structure of such an environment 100 but further discussion of the hardware, applications programs and data that embody the methodology of the present disclosure is described elsewhere herein.

The environment 100 includes one or more data servers 104 which communicate with the cloud server 102 and delivery system 120 via communication channels, whether wired or wireless, as is well known to those of ordinary skill in the pertinent art. In a preferred embodiment, the cloud server 102 is accessed using the Internet. For simplicity, one data server 104 is shown. The data server 104 hosts multiple Web sites and houses multiple databases necessary for the proper operation of the delivery system 120 in accordance with the subject disclosure.

The cloud server 102 and the data server 104 is any of a number of servers known to those skilled in the art that are intended to be operably connected to a network so as to operably link to a plurality of other devices, clients and specialized hardware such as the delivery system 120 via a distributed computer network. Thus, the cloud server 102 and the data server 104 may service a plurality of delivery systems 120 and a delivery system 120 may communicate with a plurality of cloud servers 102 and data servers 104. In addition to the delivery system 120, users may interact with the environment using clients (not shown) such as desktop computers, laptop computers, personal digital assistants, cellular telephones, tablets, phablets and the like. Such clients allow users easier and more convenient access to the environment 100.

The environment 100 delivers information to physicians and/or medical professionals by aggregating information, such as medical studies, medical journal articles, drug efficacy studies, publications from medical websites, other medical literature and raw and transformed scientific data. The information gathered is specific to a topic that is requested by the physician or medical professional seeking the information. For example, a medical professional can request information regarding the appropriate standard of care, current best practices, proper medical procedures, proper medical techniques, proper dosage ranges for a given medication, or other similar information.

The subject technology may also utilize other technology now known and later developed. Typically, printed circuit boards, wiring, devices such as buttons, switches and lights, computers, servers, processors, software and other hardware and software will be utilized to create functional modules as part of a computer or specialized controller. Computers, servers and/or specialized controller are one or more digital data processing devices. Such a device generally can be an application specific unit, a personal computer, computer workstation (e.g., Sun, HP), laptop computer, a tablet computer, server computer, mainframe computer, handheld device (e.g., personal digital assistant, Pocket PC, cellular telephone, etc.), information appliance, printed circuit board with components or any other type of generic or special-purpose, processor-controlled device capable of receiving, processing, displaying, and/or transmitting digital data.

A typical computer or specialized controller includes random access memory (RAM), mechanisms and structures for performing input/output operations, a storage medium such as a magnetic hard disk drive(s), and an operating system (e.g., software) for execution on a central processing unit. The computer or specialized controller also has input and output devices such as buttons, lights, a keyboard, mouse, trackball and/or monitor, respectively.

A processor generally is logic circuitry that responds to and processes instructions that drive a computer or specialized controller and can include, without limitation, a central processing unit, an arithmetic logic unit, an application specific integrated circuit, a task engine, and/or any combinations, arrangements, or multiples thereof. Software or code generally refers to instructions which, when executed on one or more digital data processing devices, cause interactions with operating parameters, sequence data/parameters, database entries, network connection parameters/data, variables, constants, software libraries, and/or any other elements needed for the proper execution of the instructions, within an execution environment in memory of the digital data processing device(s).

A module is a functional aspect, which may include software, application specific hardware and/or generally available hardware configured to accomplish specific tasks and functions. Typically, a module encompasses the necessary components to accomplish a task. It is envisioned that the same hardware could implement a plurality of modules and portions of such hardware being available as needed to accomplish the task. Those of ordinary skill will recognize that the software and various processes discussed herein are merely exemplary of the functionality performed by the disclosed technology and thus such processes and/or their equivalents may be implemented in commercial embodiments in various combinations without materially affecting the operation of the disclosed technology.

Still referring to FIG. 1, the environment 100 for executing methods in accordance with the subject technology includes a fabrication module 122 that is part of the delivery system 120. The delivery system 120 may be housed in a single location or, more typically, include various components in a plurality of locations.

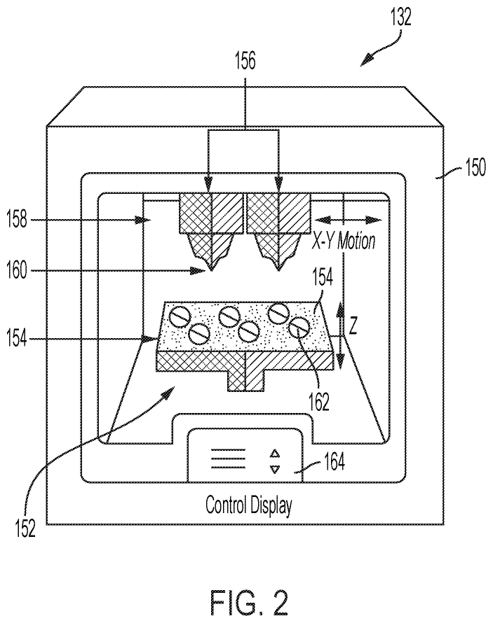

The fabrication module 122 includes a processor 124 that is connected to the Internet or another network connection. In FIG. 2, the processor 124 communicates with the cloud server 102 and the data server 104 so that searching and storing of information in databases can be performed as needed. The processor 124 is connected to a power source 126 such as a wall outlet. The processor 124 is in communication with memory 128 so that data, algorithms, instructions and the like that are stored on the memory 128 can be searched and utilized to perform the subject methods.

To output the information to a medical professional, the delivery system 120 includes a display 130 such as a monitor and/or printer. The deliver system 120 can also include integrated and/or stand-alone dispensers 132 (only one shown for simplicity). In one embodiment, the dispenser 132 is a 3D printer as shown in FIG. 2. Still referring to FIG. 1, a transmitter/output port 134 transmits files, instructions and the like between the processor 124 and dispenser 132. The display 130 provides an interface for the patient, medical professional or pharmacist to review information and interaction with the delivery system 120. Again, the delivery system 120 may include card readers for accepting payment, and a host of buttons such as shown on a touch screen monitor, to ease interaction. The transmitter/output port 134 is capable of transmitting the information to another device, such as a portable electronic device, a cell phone, a tablet, a laptop computer, a desktop computer, or another device. The transmitter in all of the above cases can be configured as a transmitter/receiver, so that the processor can receive information or commands from another device.

Referring to FIG. 2, a schematic representation of the 3D printer 132 in accordance with the subject technology is shown. The 3D printer 132 may be specially designed and adapted as the dispenser 132 of the delivery system 120. The 3d printer 132 includes a housing 150 having a sterile interior 152. A stage 154 is free to move in the Z axis. A printer head 156 that is free to move in the X and Y axis includes a syringe component 158 for drawing a source material (not shown) into the printer head 156. When the source material is extruded or otherwise released from replaceable print tips 160, a product 162 such as pill can be formed on the stage 154. Users interact with the 3D printer 132 by using a combination controller/display panel 164. In one embodiment, the 3D printer 132 includes specialized hardware to perform all the modules and functions of the entire delivery system.

The flowcharts herein illustrate the structure or the logic of the present technology, possibly as embodied in computer program software for execution on a computer, digital processor or microprocessor. Those skilled in the art will appreciate that the flow charts illustrate the structures of the computer program code elements, including logic circuits on an integrated circuit, that function according to the present technology. As such, the present technology may be practiced by a machine component that renders the program code elements in a form that instructs a digital processing apparatus (e.g., computer) to perform a sequence of function step(s) corresponding to those shown in the flow charts.

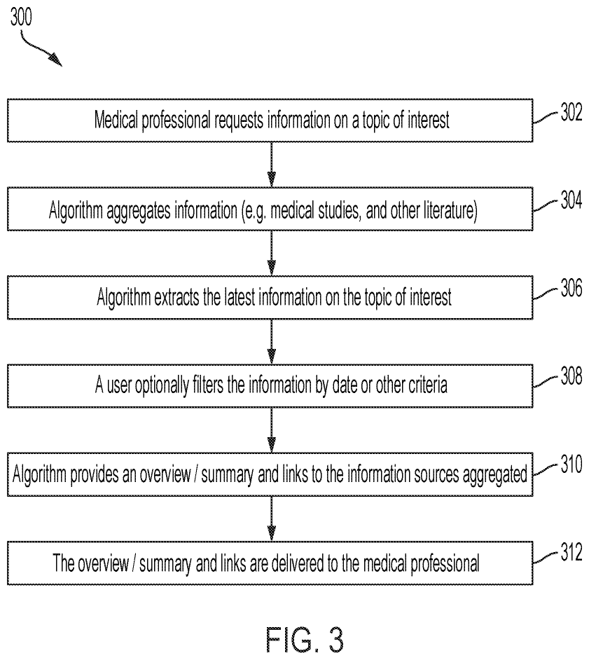

Referring now to FIG. 3, there is illustrated a flowchart 300 depicting a research process for finding the most pertinent information for health care providers and patients in accordance with an embodiment of the present technology. By virtue of the Internet, subscription sources, information stored in the cloud server 102, the data server 104, and the memory 128, the environment 100 has access to peer reviewed research, large scale studies and the like so that the most up-to-date information on a topic of interest can be provided. The research process is useful for providing information to medical professionals to educate them quickly. This is particularly useful for medical professionals seeking the most up-to-date information, and for medical professionals seeking information on topics (such as medical conditions) that are rare, or for medical professionals seeking information on topics that they are not accustomed to encountering.

At step 302, the medical professional requests information on a topic of interest by interaction with the delivery system 120. The delivery system 120 aggregates related information from the cloud server 102, data server 104, the memory 128, the Internet and the like at step 304. The delivery system 120 culls out the most relevant and important information on the topic of interest at step 306. At step 308, the medical professional can review and filter the relevant information by date, publication type and/or other criteria. Once the search and analysis is completed, the delivery system 120 generates an overview/summary complete with source links at step 310. At step 312, the overview/summary and links are delivered to the medical professional such as by email, text, printing and/or presentation of the display 130.

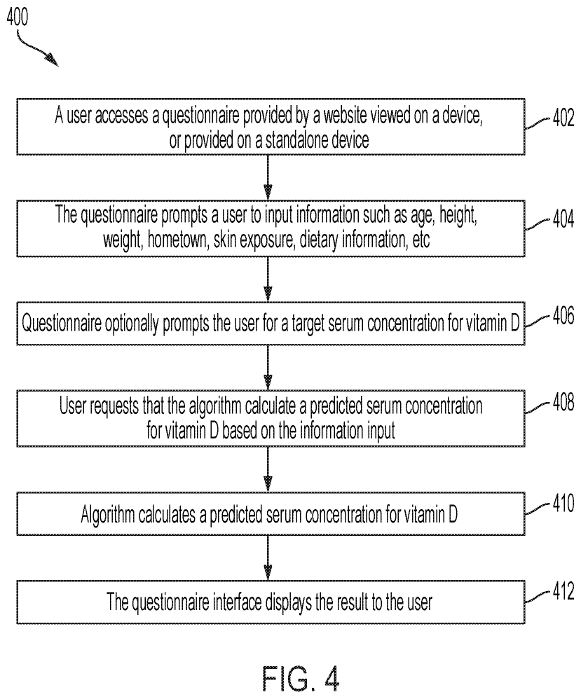

Referring now to FIG. 4, there is illustrated a flowchart 400 depicting a questionnaire process for determining a recipe for a medication in accordance with an embodiment of the present technology. In this example, the flowchart 400 relates to determining a vitamin D concentration. At step 402, the patient starts the process by accessing a questionnaire. At step 404, the delivery system 120 prompts a patient to input health data through the questionnaire on the control display 164, an Internet web page, smartphone application, or another device or method for answering the questionnaire. The patient inputs information such as age, skin exposure and the like to create a health profile. Health data for the profile can be input or downloaded with or without prompting a patient to input the data. For example, health data can be downloaded from a health tracking device such as a smartphone, pedometer, or another device. The steps of the method can be performed by a web application, smartphone application, or another application implemented on various computer devices.

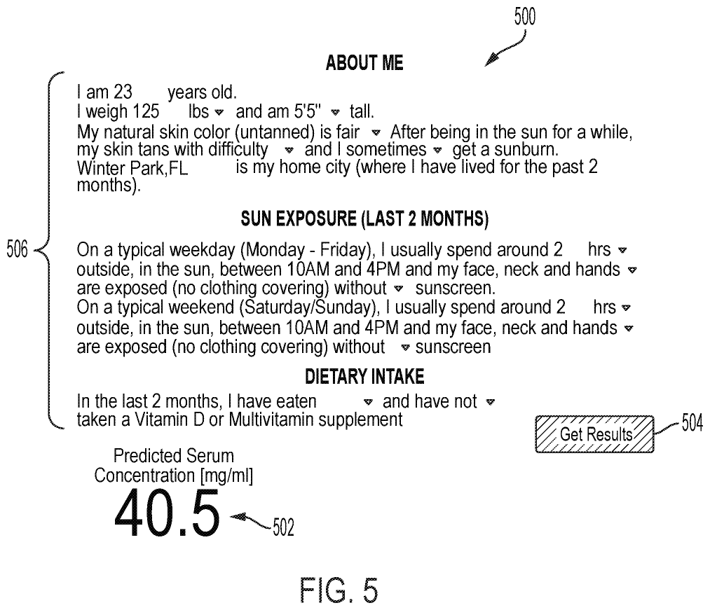

At step 406, the patient can optionally enter a target serum concentration for vitamin D. At steps 408 and 410, the delivery system 120 calculates a predicted serum concentration for that patient based on the health profile of the patient and at least one property of vitamin D when prompted to do so by the patient. The results of the calculation are presented to the patient at step 412. For example, FIG. 5 illustrates a typical results page 500 of the flowchart 400 including a result 502, a button for requesting the results 504, and a portion 506 of the health profile.

Preferably, the health profile includes important information so that the relevant factors in adjusting the dosage can be properly weighed and considered. Additionally, the information related to the medication, such as uncovered by using the process of flowchart 3, is utilized in step 410. For example, standard doses for vitamin D to prevent osteoporosis and immune dysfunction in typical people can facilitate customization of the dosage.

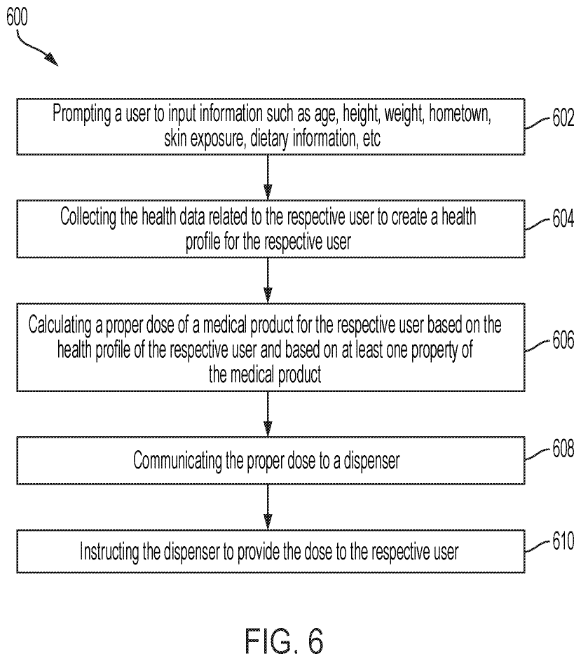

Referring now to FIG. 6, there is illustrated a flowchart 600 depicting a fabrication process for a customized medication in accordance with an embodiment of the present technology. In this example, the flowchart 600 again relates to vitamin D. Similar to above, steps 602 and 604 collect patient information to create a health profile. However, in this flowchart 600, it is likely a pharmacist entering the information. Further, the pharmacist preferably has a delivery system 120 or at least the dispenser 132 on premises.

Once the proper dose is calculated at step 606, the delivery system 120 then communicates the proper dose to the dispenser 132 at step 608, The dispenser 132, such as a 3D printer, is configured to print a pill of the vitamin, multivitamin, medication, etc. The dispenser 132 is capable of printing a range of doses that would be useful to at least a majority of consumers, based on typical vitamin D needs of individuals in the general population. The 3D printer can be provided with a bottling and capping machine so that the printed pills can be automatically packaged for a consumer. At step 610, the delivery system may provide a supply of the packaged doses with instructions to the pharmacist or patient. It is envisioned that the dispenser 132 can be owned and operated by a third party, and the dispenser 132 can be configured to receive a signal from the processor 124 regarding the proper dose to dispense, wherein dispensing can include mail delivery and auto-refill.

As can be seen, the method of flowchart 600 removes guesswork from choosing the proper vitamins or other supplement by offering a personalized dietary supplement to help each individual achieve his or her optimal health. The present disclosure provides doses that are “just right” for individuals, so that each dose is not too high or too low for the respective individual. The targeted dose for the individual prevents harm to that individual that would result from an incorrect dose. While vitamin D is discussed primarily herein, the method and system of the present disclosure can be applied to vitamin D or pharmaceuticals such as Adderall and the like. For example, the present disclosure could provide a custom multivitamin to an individual. The method and system can also be used to provide custom doses for the top 100 prescription medications where customized doses will increase efficacy and decrease side effects the most.

The subject technology can be used by a third party, such as pharmacists, who could produce their own custom pills. This extends new value to not only the consumers who are finally receiving the proper dose, but also to the distributors as the distributors will have a method of differentiating themselves from their competition. Pharmacies can provide individuals with prescription medication and the individual can request to have custom vitamins made while shopping in the pharmacy. Alternatively, a patient's doctor could even request specific dosing from the system of the present disclosure produced by a partner pharmacy.

It is envisioned that questionnaires would be used to intake patient information. For example, the patient's gender, age, residence, weight, skin color among other factors would be used in determining proper dosage depending upon the relevant medication. Preferably, the delivery system 120 prompts the patient for health information. The deliver system 120 can also intake activity data (e.g., typical steps per day), habit data (e.g., typical sleep hours per day), and health data (e.g., heartrate) from tracking devices such as smartphones, pedometers or other exercise/activity tracking devices.

Preferably, the delivery system 120 provides a report detailing the patient's outlook (e.g., predicted current vitamin D level, or other vitamin level) based on the data collected and how much of a supplement is needed to achieve optimum levels. In one embodiment, the information is instantly analyzed. Finally, if the patient is deficient and chooses to order a supplement, the supplement or medication is shipped every 30 days, monthly, or any other predetermined time period as recommended.

In another embodiment, the delivery system 120 analyzes not only the human parameters (e.g., such as age, gender skin color) but fits the parameters to a mathematical model that is based on decades of clinical studies and data points from tens of thousands of people all over the world. The personalized dosage may be determined without the need for a blood test. The delivery system 120 is continually working to gather relevant data and updating the cloud server 102 and the data server 104. As a patient's lifestyle changes (e.g., she moves), so do the parameters that affect her vitamin D level with a corresponding adjustment in dosage.

For example, a person from the Northeast who recently traveled to Florida for a few days, would have a normal vitamin D level if they had a blood test over the next few weeks. But a month later that person would be deficient. By using regular data from health trackers and smart phones to update a patient's profile, it is possible to ensure customers are receiving monthly supplements tailored to their needs. Not only is this more cost effective than existing blood tests, it can also operate synergistically with blood tests and doctor visits. In fact, the present disclosure allows doctors to be involved in the process. Generally, at most blood tests are on an annual basis, whereas the delivery system 120 is constantly monitoring and taking environmental considerations into account. A blood test is only a snapshot in time. In contrast, the present disclosure provides a more complete picture.

Preferably, the overall composition (fillers and binders) and shape of the tablets are preferably optimized for the vitamin or medication. In one embodiment, the step of calculating the proper dose of the medical product relies on the latest information regarding the topic of interest. For example, the calculation of the proper dose of vitamin D for an individual can rely on the latest medical journal articles regarding proper vitamin D doses. The delivery system 120 may also prompt a physician to develop a treatment plan for a patient.

Continuing with the vitamin D example, the delivery system 120 may include a mathematical model that estimates the amount of vitamin D that an individual should have in their body. The first step of the mathematical model determines a baseline (expected value) of blood serum level of vitamin D that a person should have, based on the individual's cohort. This baseline is then adjusted, based on a variety of factors such as age group, gender, race/skin color, latitude, season, etc. The delivery system 120 determines the impact of each of these characteristics on the expected baseline. The method applies a regression equation (which may vary by cohort) for a given characteristic of the individual. The method outputs a blood serum level delta, which represents the difference by which the baseline blood serum level should be adjusted for that respective characteristic. The blood serum level can be measured by the (OH)D serum concentration, for example. The delivery system 120 applies all of the respective deltas for the respective characteristics to the baseline for the individual to obtain an expected value for that particular individual.

Ranges for Caucasian, African Americans, Latinos, etc. can be included. The population baseline can serve as an initial starting point for an individual's vitamin D level which can be adjusted based on the individuals activities including sun exposure, supplementation and diet. Individual characteristics of a person and the level of sun exposure of the individual affect that person's vitamin D) level, and the impact of the characteristics can be measured by blood tests. These characteristics and other factors affecting vitamin D) level in response to sun exposure include the current time of year, the current time of day, the length of time spent in the sun, latitude of the person's residence, percent of the person's body exposed to the sun, skin type, and sunscreen application. Regressions can be performed on these characteristics and on the individual's body mass index (BMI), which is the individual's height divided by the individual's weight.

An equation is used to calculate the level of sun exposure for an individual. The sun exposure level depends on personal characteristic of the patient, their location (e.g., where they reside), their daily activity, their lifestyle factors, clothing, and sunscreen, and the preexisting serum level of vitamin D. Daily, hourly, or predicted UV and solar radiation data is gathered or extrapolated from available weather or scientific data collection sites. The patient's location is then used to determine the geographically closest data collection site. The patient's location can be provided, in some cases automatically by an electronic device such as a cell phone or a pedometer or another device, or manually entered.

The delivery system 120 provides a diagnostic tool for grading sun exposure. The sun exposure an individual receives is determined as well as the amount of sun received which could be used by the body to produce vitamin D. Sun exposure is graded on factors including intensity, area of skin exposed, and time exposed. Actual exposure can also be tracked by a dosimeter, sensor or other data collection device for use in the calculations and/or generation of a typical profile.

Given the calculated level of sun exposure based on personal characteristics and UV and solar radiation data, the amount of vitamin D produced in response to an individual's sun exposure can be determined. Personal characteristics including age, skin type, and BMI, determined are referenced to determine the amount of solar energy required for the individual to generate vitamin D. The individual data and solar spectra data would be compared to determine past, current and predicted future values for the available solar energy to vitamin D conversion. The comparison of this allows the determination of Vitamin D levels in response to sun exposure as well as a baseline vitamin D level based on sun exposure as well as other factors like diet and biometrics. By comparing the output of the mathematical model to the individual's desired/ideal level of vitamin D, the optimal vitamin D supplement level for that individual can be calculated.

Another particularly beneficial application of the subject technology is in pediatric vitamins and medications. The dispenser 132 can be adapted to fabricate medication in gummy form. The dispenser 132 would be a 3D printer with interchangeable pods containing the gummy gel components, flavoring components and active pharmaceutical ingredient (API).

In one embodiment, the API (e.g., prednisolone) is provided by a compounding pharmacist, who mixes the API with the Vitae gummy formulation base in a cartridge that is used by the 3D printer to produce the desired flavored gummy medication. Because the pharmacist performs the compounding step, and the initial printer pods will not include the drug itself, the dispenser 132 is considered pharmacy equipment similar to a capsule filling device. Hence, the dispenser 120 will not require FDA approval.

In another embodiment, the dispenser 132 includes pods containing the drug, and will require FDA approval. Such a dispenser 132 can easily transition between pods including the API and pods without. 3D printing of gummy formulations for pediatric drugs will offer rapid production of custom doses and drug release profiles in a palatable format that should increase medication adherence in a hard-to-treat population.

For solid tablets, 3D printing is useful for drugs that need age and/or weight based dosing due to toxicity or side effects, and for custom dosing forms. The dispenser 132 addresses medical non-adherence in young children, is designed for use in individual compounding and hospital pharmacies, is fully automated for cost-effective use, and provides validation and documentation for each batch produced.

The gummy size and shape are determined based upon the desired dose. For example, a plurality of various size bear-shapes can be selected to provide a range of doses. If the dose exceeds the range, multiple shapes can be utilized or a simple form (e.g., a cube) can be used to provide the desired dose. For example, the cross-sectional shape is rectangular with an adjustable height to provide the specified dose.

Various pods can provide different flavors and sweetness so that dose flavor can be based on feedback from each parent/child. The flexibility of the delivery system 120 will enable the easy titration of doses, and also the combination of several compatible therapeutics into a single dosage form. Automating and reducing time for individualized preparations will increase accessibility and reduce costs for custom dosing. After fabrication, the delivery system 120 evaluates the mass of each gummy to ensure proper amount of gel is extruded.

In one embodiment, the chemical formula is based on a combination of the seaweed-derived anionic polysac-charide sodium alginate and CaCl₂. Alginate gels have been widely studied for the delivery of small molecule drugs and the roughly 5 mm pore size leads to rapid diffusion of drug throughout the matrix. Alginate (C269C4G2ZQ) is deemed biocompatible by the FDA and is approved for use in oral drugs including tablets, oral suspensions, lozenges, and troches. For example, sodium alginate is used in some loratadine and aspirin tablets and in foods such as pastry fillings and dairy products. Moreover, unlike the animal-derived gelatin that is used to make most gummy candies, alginate is plant-derived and therefore vegan. CaCl₂ provides a divalent ion to crosslink the alginate to form a thick polymer hydrogel that can be extruded at room temperature, which will help maintain integrity with an API like prednisolone.

Premeasured volumes of sodium alginate solution are mixed with a saturated CaCl₂ solution, and the final product is shaped by the dispenser 132 during an extrusion process. Currently, compounding pharmacies produce gummies by heating gelatin. The dispenser can reduce heat-related risk to the API by using a room-temperature reaction. Alginate and CaCl₂ concentrations are optimized to provide the best balance of quick setting and drying at room temperature without damaging the prednisolone. Preferably, the dispenser 132 contains separate reservoirs for sodium alginate (with the API dissolved in) and CaCl₂, which will be combined during the printing process in a static mixing nozzle to produce the gel and incorporate the prednisolone. The size of the gummy will determine the dose, as the alginate/prednisone ratio will be held constant.

Taste is important to improving pediatric compliance. Flavor preferences vary greatly due to factors such as age and cultural and ethnic background. The ability to adjust flavor easily for different patients is critical for medication adherence for a drug as important but unpalatable as prednisolone. A sweetened flavor liquid is added to the CaCl₂ reservoir. The CaCl₂ cartridge will be similar across different APIs so that different flavors can be selected without reformulation. Predefined small quantities of flavoring will be mixed with the gel formulation during the crosslinking process. The flavor compartment is disposable and easily replaced to prevent cross contamination and to allow new flavors to be used with existing reservoirs for the chemical formulation.

The ultimate formulations will be based upon optimizing taste, drying time, ductility and texture, viscosity and the “beyond use” date (BUD). Carbmellose/carbomer gels and the like can also be utilized as well as excipients such as microcrystalline cellulose may be added to adjust release profiles as needed. A dusting of corn starch or powdered sugar can be used to prevent sticking if the gummies are overly tacky. An additional step may be necessary to rinse off any remaining sodium from the gummies. These gummies, as with current hand-compounded gummies, will be stored in the refrigerator. Each gummy may need to be packaged individually based on the tackiness and day-to-day stability.

It can therefore be seen that the present disclosure provides a method and/or system for improving the quality of care provided by medical professionals to patients; provides a method and/or system for aggregating the latest medical information and data regarding a topic of interest, and delivering that information to a doctor; provides a method and/or system for calculating the proper dose of a medical product for a patient, and dispensing the proper dose of the medical product to the patient; provides a method and/or system for aggregating the latest medical information regarding a topic of interest, acquiring information regarding a patient profile, using the latest medical information, data and patient profile to calculate the proper dose of a medical product for the patient, and dispensing the proper dose of the product to the patient; and provides a method and/or system for educating medical professionals regarding the standard of care, best practices, proper dosages, and proper medical procedures. For these reasons, the instant disclosure is believed to represent a significant advancement in the art which has substantial commercial merit.

While there is shown and described herein certain specific structure embodying the disclosure, it will be manifest to those skilled in the art that various modifications and rearrangements of the parts and steps may be made without departing from the spirit and scope of the underlying inventive concept and that the same is not limited to the particular forms herein shown and described except insofar as indicated by the scope of the appended claims.

1-15. (canceled)

16. A delivery system communicatively linked to a cloud server, the delivery system comprising:
a fabrication module, the fabrication module comprising:
a processor, the processor communicatively linked to at least the cloud server and a data server;
a power supply, the power supply linked to the processor;
a memory, the memory linked to the processor;
a display, the display linked to the processor;
a transmitter/output port, the transmitter/output port linked to the processor; and
a dispenser, the dispenser linked to the transmitter/output port,
the processor including a process to aggregate related information of a topic of interest from the cloud server, the data server and the memory, cull out the most relevant and important information on the topic of interest, and generate an overview/summary report on the topic of interest.

17. The delivery system of claim 16 wherein the transmitter/output port transmits files, instructions and the like between the processor and the dispenser.

18. The delivery system of claim 17 wherein the dispenser is a three dimensional (3D) printer.

19. The delivery system of claim 17 wherein the display provides an interface for a patient, a medical professional or a pharmacist to review information and interaction with the delivery system.

20. The delivery system of claim 19 wherein the generated overview/summary report on the topic of interest is cast on the display.

21. A delivery system comprising:
a processor, the processor communicatively linked to at least a cloud server and a data server;
a memory, the memory linked to the processor;
a display, the display linked to the processor;
a transmitter/output port, the transmitter/output port linked to the processor; and
a dispenser, the dispenser linked to the transmitter/output port, the processor including a process to aggregate information regarding at least one medical topic of interest, extract and filter the aggregated information, and deliver a subset of the information filtered to a healthcare professional based upon the extracting and filtering.

22. The system of claim 21 wherein the information comprises at least one of a standard of care, best practices proper dosage and proper procedures.

23. The system of claim 22 wherein the information further comprises at least one of medical studies and medical literature.

24. The system of claim 23 wherein the process further includes instructing a physician on developing a treatment plan for a patient based on the subset of information delivered.
